(19) United States
(12) Patent Application Publication
(10) Pub. No.: US 2023/0151397 A1
(43) Pub. Date: May 18, 2023

(54) Method and Equipment of Phenethylamine Production

(71) Applicant: ENZYMASTER (NINGBO) BIO-ENGINEERING CO., LTD., Ningbo (CN)

(72) Inventors: Zhuhong YANG, Ningbo (CN); Zikun WANG, Ningbo (CN); Yong Koy BONG, Ningbo (CN); Jiyong WANG, Ningbo (CN); Baoqin CAI, Ningbo (CN); Ying ZHU, Ningbo (CN); Jianmin LIU, Ningbo (CN); Zhipeng MA, Ningbo (CN); Baohua ZHANG, Ningbo (CN); Huaihao ZHU, Ningbo (CN); Marco BOCOLA, Ningbo (CN); Yuming LV, Ningbo (CN); Baoguo SUN, Ningbo (CN); Zhenlin LV, Ningbo (CN)

(73) Assignee: ENZYMASTER (NINGBO) BIO-ENGINEERING CO., LTD., Ningbo (CN)

(21) Appl. No.: 17/917,943

(22) PCT Filed: Apr. 3, 2021
(86) PCT No.: PCT/CN2021/085499

§ 371 Date: Oct. 10, 2022

(30) Foreign Application Priority Data
Apr. 10, 2020 (CN) 202010283989.7

(51) Int. Cl. C12P 13/00; C12M 1/26; C12M 1/00; C12M 1/40

(52) U.S. Cl. CPC C12P 13/001; C12M 33/10; C12M 29/18; C12M 21/18

(57) ABSTRACT

A production equipment of phenethylamine includes a first reaction device; a second reaction device, wherein the second reaction device is connected to the first reaction device; a circulation device, wherein the circulation device is respectively connected to the first reaction device and the second reaction device; an acetone storage device; a centrifugal extraction device; a phenethylamine processing device; and a phenethylamine storage device. The first reaction device and the second reaction device have the same structure. A production method of phenethylamine is further provided, which uses transaminase as a catalyst, so that acetophenone and isopropylamine can flow through the transaminase to complete the reaction, the reaction is completed in one step, the production cycle is shortened, and the production cost is reduced.

### CROSS REFERENCE TO THE RELATED APPLICATIONS

This application is the national phase entry of International Application No. PCT/CN2021/085499, filed on Apr. 3, 2021, which is based upon and claims priority to Chinese Patent Application No. 202010283989.7, filed on Apr. 10, 2020, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBBJJL001_Sequence Listing.txt, created on Sep. 28, 2022, and is 4,645 bytes in size.

### TECHNICAL FIELD

The present invention relates to the technical field of pharmaceutical intermediates, and specifically relates to a production method of phenethylamine, and also relates to a production equipment of phenethylamine.

### BACKGROUND

R-(+)-1-phenethylamine is an important pharmaceutical intermediate., with a global market demand of several thousand tons every year. The existing production process uses acetophenone as the starting material which is hydrogenated with liquid ammonia using nickel metal catalysis to prepare racemic phenethylamine under high temperature and high pressure. The reaction conditions are quite severe, flammable and explosive, which puts high demands for equipment and operators. An equivalent amount of resolution reagent is used to resolve racemic phenethylamine in order to obtain R enantiomer. The resolution efficiency is low and the yield is only 30%, the ee value of the product is limited by technology itself and can only reach 98%. A large amount of inorganic acid is used, and then sodium hydroxide is used for neutralization in the resolution process, resulting in a large amount of waste salt and wastewater.

CN1226228A disclosed a chemical method for preparing racemic phenethylamine, and a resolution reagent (acid) was used to finally obtain chiral phenethylamine. The disadvantages of this method were obvious: the complex multi-step reaction, the introduction of a resolution reagent and the generation of a large amount of waste acid, which is prone to environmental pollution problems.

CN103641724A disclosed a method for synthesizing phenethylamine, which used highly toxic organic compounds such as phenylacetamide, zinc borohydride, tetrahydrofuran, toluene. etc. It involves multi-step reactions under high temperature and pressure conditions to obtain chiral phenethylamine.

CN101337898A disclosed a meta-hydroxyphenethylamine, which is prepared by catalytic hydrogenation reduction to obtain meta-hydroayphenethylamine. The disadvantage of this method is the dangerous operation of catalytic hydrogenation, and it is also a multi-step reaction.

In order to solve the above problems, there has been a method of using transaminase as a catalyst to increase the yield of R-(+)-1-phenethylamine.

Transaminase, also known as aminotransferase can catalyze the transfer of the amino group from the amino donor to the prochiral ketone acceptor to obtain chiral amine product and by-product ketone. The reaction process requires pyridoxal phosphate.

However, in actual application, the use of transaminase has the following shortcomings: it is difficult to reuse transaminase; the presence of residual protein in the reaction solution.

Therefore, there is an urgent need for a phenethylamine production method. and equipment that can recycle transaminase in order to reduce reaction time as well as production cost.

### SUMMARY

One of the aims of the present invention is to provide a production equipment of phenethylamine for solving, the existing shortcomings.

In order to achieve the above aim, the technical solutions provided by the present invention are as following.

A production equipment of phenethylamine, including:

A first reaction device, which is set with transaminase;

A second reaction device, which is connected to the first reaction device, and the second reaction device is set with transaminase;

A circulation device, which is respectively connected with the first reaction device and the second reaction device;

An acetone storage device, which is connected to the circulation device;

A centrifugal extraction device, which is connected to the circulation device;

A Phenethylamine processing module, which is connected to the centrifugal extraction device;

A Phenethylamine storage device, which is connected to the phenethylamine processing module;

Wherein, the first reaction device and the second reaction device have the same structure.

Preferably, the phenethylamine processing module includes:

A phenethylamine inorganic acid salt separation device, which is connected to the centrifugal extraction device;

A neutralization device, which is connected to the phenethylamine inorganic acid salt separation device;

A Phenethylamine device, which is respectively connected with the neutralization device and the phenethylamine storage device.

Preferably, the production equipment of phenethylamine further includes:

An acetophenone separation device, which is connected to the centrifugal extraction device;

An acetophenone recovery device, which is connected to the acetophenone separation device.

Preferably, the production equipment of phenethylamine further includes:

A dichloromethane separation device, which is respectively connected with the phenethylamine inorganic acid salt separation device and the acetophenone recovery device;

A dichloromethane recovery device, which is connected to the dichloromethane separation device.

Preferably, the production equipment of phenethylamine further includes;

An alkali storage device, which is respectively connected with the neutralization device and the acetophenone separation device.

Preferably, the production equipment of phenethylamine further includes:

An acid storage device, the acid storage device is connected to the centrifugal extraction device.

Preferably, the first reaction device includes:

Main body;

A first filter plate, which is set in the upper part of the inside of the main body;

A second filter plate, which is set in the lower part of the inside of the main body;

An orifice plate, which is arranged at the lower part of the first filter plate;

A number of water cap distributing elements, which are respectively arranged on the lower side of the first filter plate and the upper side of the second filter plate.

Preferably, the production equipment of phenethylamine further includes:

At least one of a third reaction device, whereas the said third reaction device is respectively connected with the first reaction device and the second reaction device, and the third reaction device is set with transaminase;

Preferably, there are several third reaction devices; the first reaction device, the third reaction devices, and the second reaction device are connected in sequence; the first reaction device is connected to a few of the third reaction devices.

Preferably, the production equipment of phenethylamine further includes:

A first condensing device, which is respectively connected with the circulation device and the acetone storage device.

Preferably, the production equipment of phenethylamine further includes:

A second condensing device, which is respectively connected with the phenethylamine distillation device and the phenethylamine storage device.

Preferably, the production equipment of phenethylamine further includes:

A third condensing device, which is respectively connected with the dichloromethane separation device and the dichloromethane recovery device.

The second aim of the present invention is to provide a production method of phenethylamine in view of the deficiencies in the prior art.

In order to achieve the above aims, the technical solutions provided by the present invention are as following.

A production method of phenethylamine, comprising the following steps:

Step S1: acetophenone, isopropylamine and pyridoxal phosphate are supplied to the circulation device;

Step S2: the acetophenone, the isopropylamine and the pyridoxal phosphate are circulated several times in the circulation loop formed by the circulation device, the first reaction device and the second reaction device; after the circulation, acetone and the crude product of phenethylamine are produced;

Step S3: the circulation device transports the acetone to the acetone storage device for storage, and transports the crude phenethylamine to the centrifugal extraction device;

Step S4: with the action of inorganic acid, the crude phenethylamine is processed in the centrifugal extraction device to obtain the recovered acetophenone and phenethylamine inorganic acid salt:

Step S5: the centrifugal extraction device transports the recovered acetophenone to the acetophenone recovery device for storage, and transports the phenethylamine inorganic acid salt to the phenethylamine processing module.;

Step S6: with the action of dichloromethane and alkali sequentially, the crude phenethylamine is processed in the phenethylamine processing module to obtain recovered acetophenone and phenethylamine respectively;

Step S7: the phenethylamine processing module transports the phenethylamine to the phenethylamine storage device for storage;

Wherein, the first reaction device and the second reaction device are stored with transaminase.

Preferably, the amino acid sequence of the transaminase is shown as SEQ ID NO: 2.

Preferably, in the step S4, an acid storage device transports the inorganic acid to the centrifugal extraction device.

Preferably, in the step S4 the inorganic acid is a concentrated inorganic acid.

Preferably, the mass fraction of the concentrated inorganic acid is greater than 75%.

Preferably, in the step S4, the inorganic acid is a dilute inorganic acid.

Preferably, in the step S4, the inorganic acid is sulfuric acid or concentrated hydrochloric acid.

Preferably, for the step S3 and the step S4, it may also be:

Step S3: the circulation device transports the crude phenethylamine and acetone to a buffer device, and then transports inorganic acid to the buffer device. After reaction under certain conditions, acetone and a liquid phase are obtained, and then the buffer device transports the acetone to the acetone storage device for storage, and transports the liquid phase to the centrifugal extraction device;

Step S4: the liquid phase is processed in the centrifugal extraction device to obtain recovered acetophenone and phenethylamine inorganic acid salt.

Preferably, in the step S5, it further comprises:

Step S51A: the centrifugal extraction device transports the recovered acetophenone to the acetophenone separation device;

Step S51B: under the action of alkali, the recovered acetophenone is processed in the acetophenone separation device to obtain inorganic sodium salt wastewater and acetophenone respectively;

Step S51C: the acetophenone separation device transports the acetophenone to the acetophenone recovery device for storage.

Preferably, in the step S51B, an alkali storage device transports the alkali to the acetophenone separation device.

Preferably, the alkali is sodium hydroxide.

Preferably, in the step S6, it further comprises:

Step S61; with the action of dichloromethane, the crude phenethylamine is processed in the phenethylamine inorganic acid salt separation device to obtain an aqueous solution of phenethylamine inorganic acid salt, a mixture of dichloromethane and acetophenone, respectively;

Step S62: the phenethylamine inorganic acid salt separation device transports the aqueous solution of phenethylamine inorganic acid salt to the neutralization device, and the mixture of dichloromethane and acetophenone is transported to the dichloromethane separation device;

Step S63: with the action of an alkali, the aqueous solution of the phenethylamine inorganic acid salt is processed in the neutralization device to obtain crude phenethylamine, and inorganic sodium salt wastewater respectively;

Step S64: the neutralization device transports the crude phenethylamine to the phenethylamine distillation device;

Step S65: the crude phenethylamine is processed in the phenethylamine distillation device to obtain a finished product of phenethylamine;

Step S66: the phenethylamine distillation device transports the finished product of phenethylamine to the phenethylamine storage device for storage.

Preferably, in the step S62, it further comprises:

Step S621A: the mixture of dichloromethane and acetophenone are processed in the dichloromethane separation device to obtain dichloromethane and acetophenone, respectively;

Step S621B: the dichloromethane separation device transports the dichloromethane to the dichloromethane recovery device for storage, and transports the acetophenone to the acetophenone recovery device for storage.

Preferably, in the step S63, an alkali storage device transports the alkali to the neutralization device.

Preferably, the alkali is sodium hydroxide.

The present invention providing the abovementioned technical solutions, compared with the prior art, has the following technical effects.

The method and equipment of phenethylamine production in the present invention use transaminase as a catalyst, so that the reaction is completed by flowing acetophenone and isopropyiamine by the transaminase. The reaction is completed in one step, which shortens the production cycle and reduces production cost; the cyclic flow reaction allows the transaminase to be reused, which further reduces production cost; acetophenone can be recycled, which reduces the discharge of liquid waste during the production process, and is environmentally friendly; the enzyme-catalyzed reaction with water as the solvent is mild, avoiding the dangerous operations such as high temperature and high pressure and catalytic hydrogenation in current chemical processes; the use of transaminase to catalyze the reaction eliminates the need for resolution reactions, which avoids the introduction of resolution agents and reduces the production of waste acid.

### BRIEF DESCRIPTION OF THE DRAWINGS

FIG. 1 is a flowchart of an exemplary embodiment of the present invention.

FIG. 2 is a cross-sectional view of the first reaction device of an exemplary embodiment of the present invention.

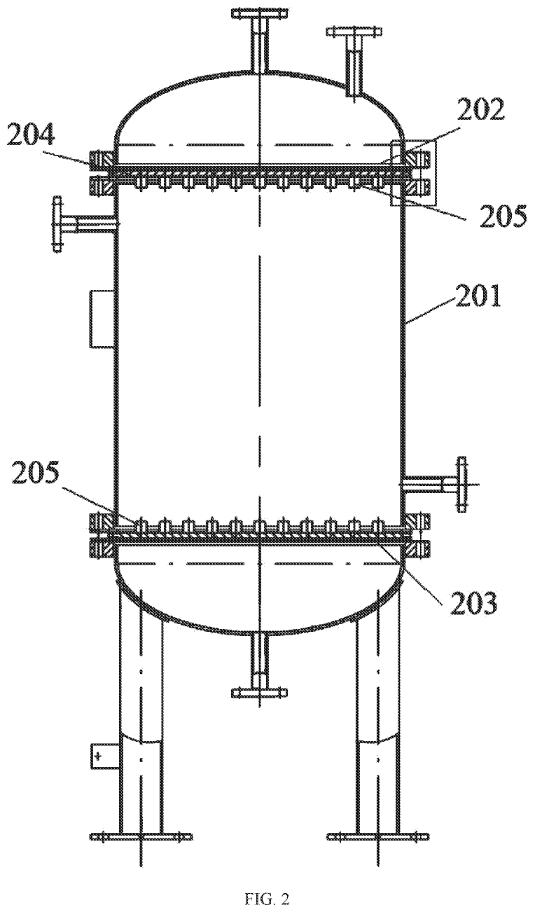

FIG. 3 is a schematic diagram of the connection relationship of the first reaction device, the second reaction device and the third reaction device of an exemplary embodiment of the present invention.

FIG. 4 is a flow chart of full operations of the first reaction device, the second reaction device and the third reaction device of an exemplary embodiment of the present invention.

FIG. 5 is a flowchart of partial operations of the first reaction device, the second reaction device, and the third reaction device of an exemplary embodiment of the present invention.

FIG. 6 is a flowchart of partial operations of the first reaction device, the second reaction device, and the third reaction device of an exemplary embodiment of the present invention.

The reference numbers in the figures are: The first reaction device 1, The second reaction device 2, The third reaction device 3, Circulation device 4, Acetone storage device 5, Centrifugal extraction device 6, Phenethylamine inorganic acid salt separation device 7, Neutralization device 8, Phenethylamine distillation device 9, Phenethylamine storage device 10, Acetophenone separation device 11, Acetophenone recovery device 12, Dichloromethane separation device 13, Dichloromethane recovery device 14, Alkali storage device 15, Acid storage device 16, The first condensation device 17, The second condensation device 18, The third condensation device 19, Acetophenone buffering device 20, Phenethylamine inorganic acid salt buffer device 21.

Main body 201, The first filter plate 202, The second filter plate 203, The orifice plate 204, The water cap distribution element 205.

### DETAILED DESCRIPTION OF THE EMBODIMENTS

With reference to the accompanying drawings in the embodiments of the present invention, the technical solutions of the given embodiments of the present invention will be clearly and completely described. Obviously, the embodiments described herein are only part of, not all of, possible embodiments of the present invention. Based on the embodiments of the present invention, all other embodiments obtained by those skilled in the art without creative work shall fall within the protection scope of the present invention.

It should be noted that the embodiments of the present invention and the features in the embodiments can be combined with each other if there is no conflict.

The following content further describes the present invention with reference to the drawings and specific embodiments, but it is not a limitation of the present invention.

### EXAMPLE 1

This embodiment is an exemplary embodiment of the production method and production equipment of phenethylamine in the present invention.

in the present invention, phenethylamine is R-(+)-1-phenethylamine, and the sequence of transaminase is shown in the following table.

TABLE 1
Transaminase sequence table

| | |
|---|---|
| Poly- | atggcaagca tggataaagt cttcagcggc |
| nucleo- | tattacgcgc gtcaaaaact gctggaacgc |
| tide | tctgataacc cgtttagcaa aggcatcgcg |
| SEQ ID | tacgttgaag gtaaactggt tctgccgagc |
| NO: 1 | gacgcacgta ttccgctgct ggacgaaggt |
| | tttatgcaca gcgacgcgac ctacactacc |
| | attagcgttt gggacggccg ctttttcegt |
| | ctggacgatc atctgcagcg tattctggaa |
| | tcctgcgaca aaatgcgcct gaaatttccg |
| | ctggcactga gcagcgttaa aaaaatcctg |
| | gcggaaatgg ttgcgaaatc tggtattcgc |
| | gatgcgatgg tcgaagttat tgttacccgc |
| | ggtctgaccg gcgttcgcgg tcgccaaccg |
| | gaagacctgt acaacaacaa catctacctg |
| | ctggttctgc cgtatatctg gctgatggca |
| | ccggaagatc aacgtcacgg cggcgaagca |
| | attattaccc gtaccgttcg tcgtaccccg |
| | ccgggcgcat tcgatccgac cattaaaaac |
| | tttcagtggg gcgatctgat caaaggcatg |
| | ttcgaagcag aagatcgcgg cgcaacctat |
| | ccgtttctga ccgacggcga tacccacctg |
| | accgaaggta gcggttttaa catcgtcctg |
| | gtcaaaaacg gcatcatcta caccccggat |
| | cgcggcgttc tggaaggtat tacccgtaaa |
| | agcgttattg acgttgcacg cgcaaacagt |
| | attgatattc gcctggaagt cgtcccggtt |
| | gaacaggcgt atcacagcga cgaaatcttc |
| | atgaccagca ccgctggcgg tattatgccg |
| | attaccctgc tggacggtca accggttaac |
| | ggcggtcagg taggcccgat taccaaaaag |
| | atttgggacg gctactggga gatgcattac |
| | aataacgcgt atagctttcc ggtcgattac |
| | ggtagcggttaa |
| Amino | Met Ala Ser Met Asp Lys Val Phe |
| acid | Ser Gly Tyr Tyr Ala Arg Gln Lys |
| SEQ ID | Leu Leu Glu Arg Ser Asp Asn Pro |
| NO: 2 | Phe Ser Lys Gly Ile Ala Tyr Val |
| | Glu Gly Lys Leu Val Leu Pro Ser |
| | Asp Ala Arg Ile Pro Leu Leu Asp |
| | Glu Gly Phe Met Hls Ser Asp Ala |
| | Thr Tyr Thr Thr Ile Ser Val Trp |
| | Asp Gly Arg Phe Phe Arg Leu Asp |
| | Asp Hls Leu Gln Arg Ile Leu Glu |
| | Ser Cys Asp Lys Met Arg Leu Lys |
| | Phe Pro Leu Ala Leu Ser Ser Val |
| | Lys Lys Ile Leu Ala Glu Met Val |
| | Ala Lys Ser Gly Ile Arg Asp Ala |
| | Met Val Glu Val Ile Val Thr Arg |
| | Gly Leu Thr Gly Val Arg Gly Arg |
| | Gln Pro Glu Asp Leu Tyr Asn Asn |
| | Asn Ile Tyr Leu Leu Val Leu Pro |
| | Tyr Ile Trp Leu Met Ala Pro Glu |
| | Asp Gln Arg Hls Gly Gly Glu Ala |
| | Ile Ile Thr Arg Thr Val Arg Arg |
| | Thr Pro Pro Gly Ala Phe Asp Pro |
| | Thr Ile Lys Asn Phe Gln Trp Gly |
| | Asp Leu Ile Lys Gly Met Phe Glu |
| | Ala Glu Asp Arg Gly Ala Thr Tyr |
| | Pro Phe Leu Thr Asp Gly Asp Thr |
| | Hls Leu Thr Glu Gly Ser Gly Phe |
| | Asn Ile Val Leu Val Lys Asn Gly |
| | Ile Ile Tyr Thr Pro Asp Arg Gly |
| | Val Leu Glu Gly Ile Thr Arg Lys |
| | Ser Val Ile Asp Val Ala Arg Ala |
| | Asn Ser Ile Asp Ile Arg Leu Glu |
| | Val Val Pro Val Glu Gln Ala Tyr |
| | Hls Ser Asp Glu Ile Phe Met Thr |
| | Ser Thr Ala Gly Gly Ile Met Pro |
| | Ile Thr Leu Leu Asp Gly Gln Pro |
| | Val Asn Gly Gly Gln Val Gly Pro |
| | Ile Thr Lys Lys Ile Trp Asp Gly |
| | Tyr Trp Glu Met Hls Tyr Asn Asn |
| | Ala Tyr Ser Phe Pro Val Asp Tyr |
| | Gly Ser Gly |

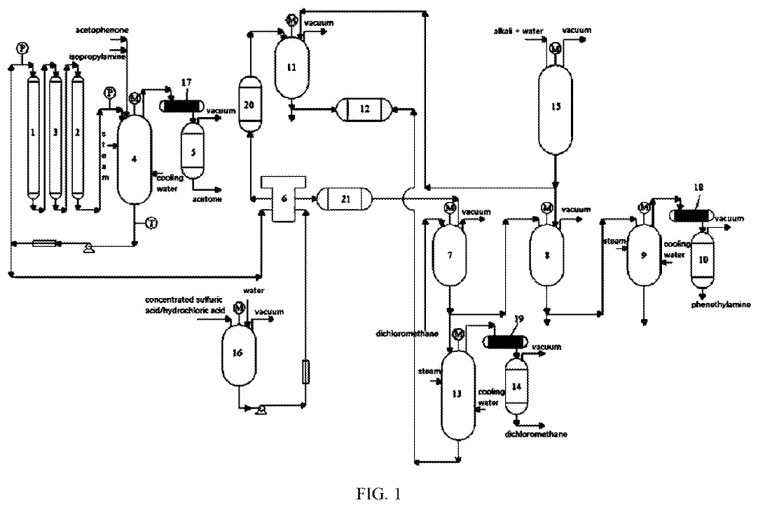

As shown in FIG. 1, the production equipment of phenethylamine, including a first reaction device 1, a second reaction device 2, a third reaction device 3, a circulation device 4, an acetone storage device 5, a centrifugal extraction device 6, the phenethylamine processing module and the phenethylamine storage device 10; the first reaction device 1, the third reaction device 3, the second reaction device 2 and the circulation device 4 are connected by pipelines to form a flow circulation loop; the circulation device 4 is respectively connected by pipelines with the acetone storage device 5 and the centrifugal extraction device 6; the centrifugal extraction device 6, the phenethylamine processing module, and the phenethylamine storage device 10 are sequentially connected by pipelines.

Wherein, the transaminase is set up at the middle chamber.

Further, the transaminase may be in the form of a free enzyme, in the form of an immobilized enzyme, or in the form of enzyme inside the recombinant expression host cell.

The structures of the second reaction device 2 and device 3 are the same as the structure of the first reaction device 1.

The number of the third reaction device 3 can be zero, one, or several, and the specific number can be set according to actual production conditions.

The circulation device 4 is respectively connected with the first reaction device 1 and the centrifugal extraction device 6 by pipelines, on which a circulation pump and a flow detection device are sequentially installed according to the material flow direction.

Further, a temperature detection device is also installed on the pipeline between the circulation pump and the circulation device 4.

Further, a pressure detection device is also installed on the pipeline between the first reaction device 1 and the flow detection device.

Further, a first condensation device 17 is also set between the circulation device 4 and the acetone storage device 5.

The phenethylamine processing module includes phenethylamine inorganic acid salt separation device 7, neutralization device 8 and phenethylamine distillation device 9. The centrifugal extraction device 6, the phenethylamine inorganic acid salt separation device 7, the neutralization device 8, the phenethylamine distillation device 9, and the phenethylamine storage device 10 are sequentially connected by pipelines.

Further, a phenethylamine inorganic acid salt buffer device 21 is also set between the centrifugal extraction device 6 and the phenethylamine inorganic acid salt separation device 7.

Further, a second condensation device 18 is also set between the phenethylamine distillation device 9 and the phenethylamine storage device 10.

Further, the centrifugal extraction device 6 is also connected to the acetophenone processing module, the said acetophenone processing module includes the acetophenone separation device 11 and the acetophenone recovery device 12. The centrifugal extraction device 6, the acetophenone separation device 11 and the acetophenone recovery device 12 are sequentially connected by pipelines.

Further, an acetophenone buffering device 20 is also set between the centrifugal extraction device 6 and the acetophenone separation device 11.

Further, the centrifugal extraction device 6 is also connected to the acid storage device 16 by pipelines, on which an acid pump and a flow detection device are sequentially installed according to the material flow direction.

Further, an alkali storage device 15 is respectively connected to the neutralization device 8 and the acetophenone recovery device 11 by pipelines.

Further, the phenethylamine inorganic acid salt separation device 7 is also connected to a dichloromethane processing module, and the dichloromethane processing module includes a dichloromethane separation device 13 and a dichloromethane recovery device 14. The phenethylamine inorganic acid salt separation device 7, the dichloromethane separation device 13, and the dichloromethane recovery device 14 are sequentially connected by pipelines.

Further, the dichloromethane separation device 13 is also connected with the acetophenone recovery device 12 by pipelines.

Further, a third condensation device 19 is also set between the dichloromethane separation device 13 and the dichloromethane recovery device 14.

The production method of phenethylamine using the above production equipment of phenethylamine includes the following steps.

Step S1: acetophenone, isopropylamine and pyridoxal phosphate are supplied to the circulation device 4;

Step S2: acetophenone and isopropylamine are circulated several times in the circulation loop formed by the circulation device 4, the first reaction device 1, and the second reaction device 2, acetone and the crude phenethylamine are obtained;

Step S3: the circulation device 4 transports acetone to the acetone storage device 5 for storage, and transports the crude phenethylamine to the centrifugal extraction device 6;

Step S4: with the action of the inorganic acid, the crude phenethylamine is processed in the centrifugal extraction device 6 to obtain the recovered acetophenone and the phenethylamine inorganic acid salt;

Step S5: the centrifugal extraction device 6 transports the recovered acetophenone to the acetophenone recovery device 12 for storage, and transports the phenethylamine inorganic acid salt to the phenethylamine processing module;

Step S6: with the action of dichloromethane and alkali sequentially, the crude phenethylamine are processed in the phenethylamine processing module to obtain recovered acetophenone and phenethylamine respectively;

Step S7, the phenethylamine processing module transports the phenethylamine to the phenethylamine storage device 10 for storage.

Preferably, in step S1, pyridoxal phosphate is also supplied to the circulation device 4.

Preferably, in step S2, the reaction formula is as follows:

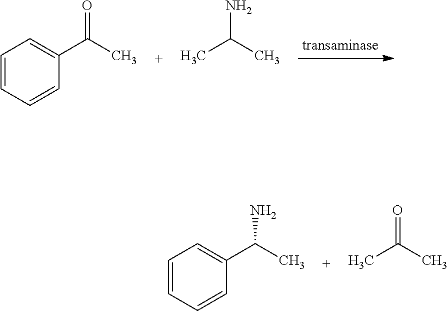

Preferably, in step S2, a third reaction device 3 may also be set in the circulation loop.

Preferably, in step S3, the circulation device 4 transports acetone to the first condensing device 17 for processing, and then to the acetone storage device 5 for storage.

Preferably, in step S4, the acid storage device 16 transports the inorganic acid to the centrifugal extraction device 6.

Preferably, the inorganic acid is a concentrated inorganic acid or a dilute inorganic acid, wherein the mass fraction of the concentrated inorganic acid is greater than 75%.

Preferably, the inorganic acid is sulfuric acid or concentrated hydrochloric acid.

Preferably, for steps S3-S4, it can also be replaced with:

Step S3: the circulation device 4 transports the crude phenethylamine and acetone to a buffer device, and the inorganic acid is transported into the buffer device. After the reaction under certain conditions, the organic phase (i.e. acetone) and the liquid phase are separated. The buffer device transports acetone to the acetone storage device 5 for storage, and transports the liquid phase to the centrifugal extraction device 6;

Step S4: the liquid phase is processed in the centrifugal extraction device 6 to obtain recovered acetophenone and phenethylamine inorganic acid salt.

Specifically, in step S3, the circulation device 4 transports the crude phenethylamine and acetone to a buffer device, and the inorganic acid is transported into the buffer device. After reacting at 50-70° C. for 1-2 hours, they are concentrated and separated at 40-60° C., the acetone is transported to the acetone storage device 5 for storage, and the liquid phase is transported to the centrifugal extraction device 6.

Preferably, in step S5, the following steps are included:

Step S51A: the centrifugal extraction device 6 transports the recovered acetophenone to the acetophenone separation device

Step S51B: with the action of alkali, the recovered acetophenone is processed in the acetophenone separating device 11 to obtain inorganic sodium salt wastewater and acetophenone respectively;

Step S51C: the acetophenone separation device 11 transports acetophenone to the acetophenone recovery device 12 for storage.

Preferably, in step S51A, the centrifugal extraction device 6 transports the recovered acetophenone to the acetophenone buffering device 20 for temporary storage, and then the acetophenone buffering device 20 delivers the recovered acetophenone to the acetophenone separation device 11 for processing.

Preferably, the alkali storage device 15 transports alkali to the acetopheriode separation device 11.

Preferably, the alkali is an aqueous solution of sodium hydroxide.

Preferably, in step S6, the following steps are included:

Step S61: with the action of dichloromethane, the crude phenethylamine is processed in the phenethylamine inorganic acid salt separation device 7 to obtain an aqueous solution of phenethylamine inorganic acid salt, a mixture of dichloromethane and acetophenone;

Step S62: the phenethylamine inorganic acid salt separation device 7 transports the aqueous solution of phenethylamine inorganic acid salt to the neutralization device 8, and the mixture of dichloromethane and acetophenone is transports to the dichloromethane separation device 13;

Step S63: with the action of alkali, the aqueous solution of phenethylamine inorganic acid salt is processed in the neutralization device 8 to obtain crude phenethylamine and inorganic sodium salt wastewater respectively;

Step S64: the neutralization device 8 transports the crude phenethylamine to the phenethylamine distillation device 9;

Step S65: the crude phenethylamine is processed in the phenethylamine distillation device 9 to obtain the finished product of phenethylamine;

Step S66: the phenethylamine distillation device 9 transports the finished product of phenethylamine to the phenethylamine storage device 10 for storage.

Preferably, in step S61, dichloromethane is used in the phenethylamine inorganic acid salt separation device 7 to wash the crude phenethylamine several times.

Preferably, in step S62, the following steps are included:

Step S621A; the mixture of dichloromethane and acetophenone is processed in the dichloromethane separation device 13 to obtain dichloromethane and acetophenone, respectively;

Step S621B: the dichloromethane separation device 13 transports dichloromethane to the dichloromethane recovery device 14 for storage, and transports the acetophenone to the acetophenone recovery device 12 for storage.

Preferably, the dichloromethane separation device 13 transports the dichloromethane to the third condensing device 19 for processing, and then transports it to the dichloromethane recovery device 14 for storage.

Preferably, in step S63, the alkali storage device 15 delivers alkali to the neutralization device 8.

Preferably, in step S66, the phenethylamine distillation device 9 transports the phenethylamine product to the second condensing device 18 for processing, and then the phenethylamine product is delivered to the phenethylamine storage device 10 for storage.

Preferably, the amounts of acetophenone, isopropylamine, pyridoxal phosphate, and transaminase used in the production process are as follows:

Acetophenone 300-1200 L;

Isopropylamine 50-240 Kg;

Pyridoxal phosphate 75-220 g;

Transaminase 30-120 Kg.

Wherein, the transaminase is in the form of transaminase-expressing wet cells, the content of effective transaminase in the transaminase-expressing wet cells is 1-20%, a further preferred effective content is 5-15%, and the most preferred effective content is 10%.

Preferably, in step S1, the mixture of acetophenone, isopropylamine and pyridoxal phosphate is preheated to 35-45° C. in the circulation device 4.

Preferably, in step S2, the circulation flow rate is 100-700 kg/h, the circulation flow rate is the flow rate flowing through the first reaction device 1, the circulation reaction time is 15-26 h, and the system pressure is 0.15-0.2 Mpa.

Preferably, in step S4, the reaction temperature of the crude phenethylamine and sulfuric acid is lower than 40° C.

Preferably, in step S4, the reaction temperature of the crude phenethylamine and concentrated hydrochloric acid is 60° C.

Preferably, in step S4, the processing temperature of the centrifugal extraction device 6 is 40-60° C.

The specific production process of the present invention is as following:

Acetopherione, isopropylamine and pyridoxal phosphate are supplied into the circulation device 4, and simultaneously steam and cooling water are supplied into the circulation device 4;

The circulation pump is turned on to circulate acetophenone, isopropylamine and pyridoxal phosphate multiple times in the circulation loop formed by the first reaction device 1, the second reaction device 2, the third reaction device 3, and the circulation device 4; the transaminase in the first reaction device 1, the second reaction device 2 and the third reaction device 3 catalyzes the reaction of acetophenone and isopropylamine to generate acetone and the crude phenethylamine;

After a certain number of cycles, the circulation device 4 transports the acetone to the first condensing device 17 for processing, and then to the acetone storage device 5 for storage;

The circulation device 4 transports the crude phenethylamine to the centrifugal extraction device 6;

Based on production needs, concentrated inorganic acid and water (distilled. water or ultrapure water) are transported into the acid storage device 16 to obtain a solution of inorganic acid at certain concentration, then the acid pump is turned on to transport the solution of inorganic acid to the centrifugal extraction device 6;

In the centrifugal extraction device 6, the inorganic acid reacts with the crude phenethylarnine to generate phenethylamine inorganic acid salt; and centrifugal extraction of the solution inside the centrifugal extraction device 6 is performed to obtain oil phase containing the recovered acetophenone and aqueous phase containing phenethylamine inorganic acid salt;

The centrifugal extraction device 6 transports the oil phase containing the recovered acetophenone to the acetophenone buffering device 20 for temporary storage, and transports the aqueous phase containing the phenethylamine inorganic acid salt to the phenethylamine inorganic acid salt buffer device 21 for temporary storage;

Based on production needs, sodium hydroxide and water (distilled water or ultrapure water) are delivered into the alkali storage device 15 to obtain a aqueous solution of sodium hydroxide at certain concentration, they are transported to the acetophenone separation device 11 and neutralization device 8 respectively;

The acetophenone buffering device 20 transports the recovered acetophenone to the acetophenone separation device 11. With the action of the sodium hydroxide aqueous solution, the recovered acetophenone is processed in the acetophenone separation device 11 to obtain inorganic sodium salt wastewater and acetophenone, the acetophenone separation device 11 transports the acetophenone to the acetophenone recovery device 12 for storage, and for further recycling;

The phenethylamine inorganic acid salt buffer device 21 transports the aqueous solution containing the phenethylamine inorganic acid salt to the phenethylamine inorganic acid salt separation device 7, and at the same time, dichloromethane is delivered to the phenethylamine inorganic acid salt separation device 7. Dichloromethane is used to wash the aqueous solution containing phenethylamine inorganic acid salt multiple times in the phenethylamine inorganic acid salt separation device 7 to obtain a aqueous solution of phenethylamine inorganic acid salt as well as a mixture of dichloromethane and acetophenone. The phenethylamine inorganic acid salt separation device 7 delivers the aqueous solution of phenethylamine inorganic acid salt to the neutralization device 8, and the mixture of dichloromethane and acetophenone are delivered to the dichloromethane separation device 13;

Steam and cooling water are supplied to the dichloromethane separation device 13, and the mixture of dichloromethane and acetophenone is processed in the dichloromethane separation device 13 to obtain dichloromethane and acetophenone, respectively;

The dichloromethane separation device 13 transports the dichloromethane to the third condensing device 19 for processing, and then dichloromethane is transported to the dichloromethane recovery device 14 for storage and for further recycling; at the same time, the dichloromethane separation device 13 transports acetophenone to the acetophenone recovery device 12 for storage and for further recycling;

In the neutralization device 8, with the action of the sodium hydroxide aqueous solution, the aqueous solution of phenethylamine inorganic acid salt is processed to obtain inorganic sodium salt wastewater and crude phenethylamine, respectively. And the neutralization device 8 transports the crude phenethylamine to the phenethylamine distillation device

The steam and cooling water are supplied to the phenethylamine distillation device 9 and the crude phenethylamine is processed to obtain the phenethylamine product and wastewater. The phenethylamine distillation device 9 transports the phenethylamine product to the second condensing device 18 for processing, and then the phenethylamine product is transported to the phenethylamine storage device 10 for storage:

Wherein, inorganic sodium salt wastewater and wastewater are transported to a wastewater processing module for processing.

The advantage of the present invention is that the usage of transaminase as a catalyst allows the reaction to be done by flowing acetophenone and isopropylamine through the transaminase. The reaction is completed in one step, which shortens the production cycle and reduces production cost; the transaminase can be reused through the cyclic flow reaction, which further reduces production cost; acetophenone can be recycled, which reduces liquid waste in the production process, and is environmentally friendly.

### EXAMPLE 2

This embodiment is an embodiment of the circulation loop of the present invention.

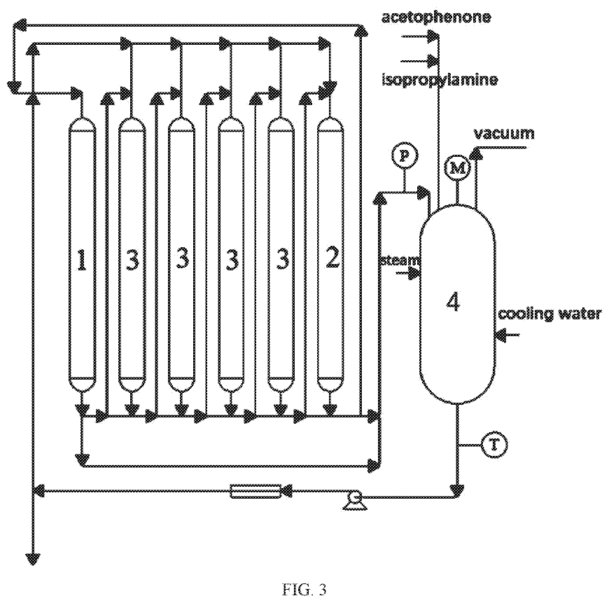

As shown in FIG. 3, the circulation loop includes a first reaction device 1, a second reaction device 2, a number of third reaction device 3, and a circulation device 4, wherein the liquid outlet pipe of the circulation device 4 is respectively connected to the liquid inlet pipe of the first reaction device 1, the liquid inlet pipe of the second reaction device 2 and the liquid inlet pipes of several third reaction device 3; and the liquid inlet pipe of the circulation device 4 is respectively connected with the liquid outlet pipe of the first reaction device 1 and the second reaction device 2; the liquid outlet pipe of the first reaction device is connected with the liquid inlet pipe of the second reaction device 2 and the liquid inlet pipes of several third reaction devices 3 respectively; liquid outlet pipe of the second reaction device 2 is connected with the liquid outlet pipe of the circulation device 4, and a plurality of valves are installed on the circulation circuit for controlling the liquid inlet direction and the liquid outlet direction.

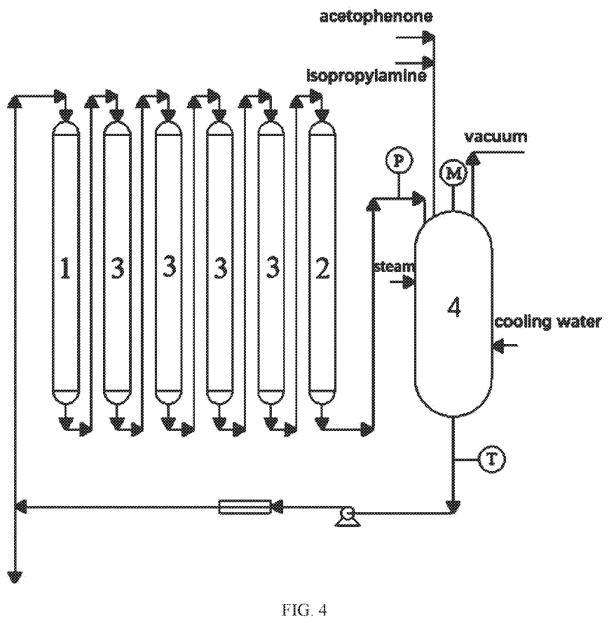

As shown in FIG. 4, when the first reaction device 1, the second reaction device 2, and several third reaction devices 3 in the circulation loop are all in a normal state, that is, all in operating states, the circulation loop is: circulation device 4→the first reaction device 1→the first one of a third reaction device 3→ . . . →the last one of a third reaction device 3→the second reaction device 2→circulation device 4.

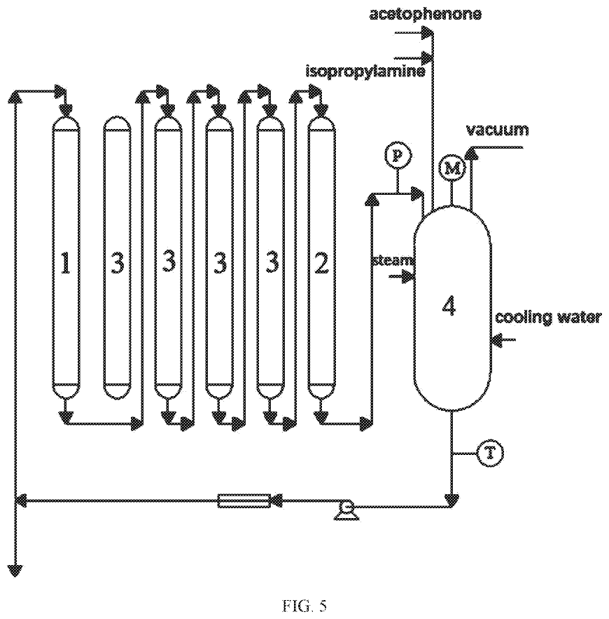

As shown in FIG. 5, when the first reaction device 1, the second reaction device 2 and several third reaction devices 3 in the circulation loop are partially in a normal state, that is, partially in operating state, the first one of a third reaction device 3 is in an overhaul state, and the corresponding valve is closed so that the material does not flow through the first one of a third reaction device 3. At this time, the circulation loop is:circulation device 4→the first reaction device 1→the second one of a third reaction device 3→ . . . → the last one of a third reaction device 3→the second reaction device 2→circulation device 4.

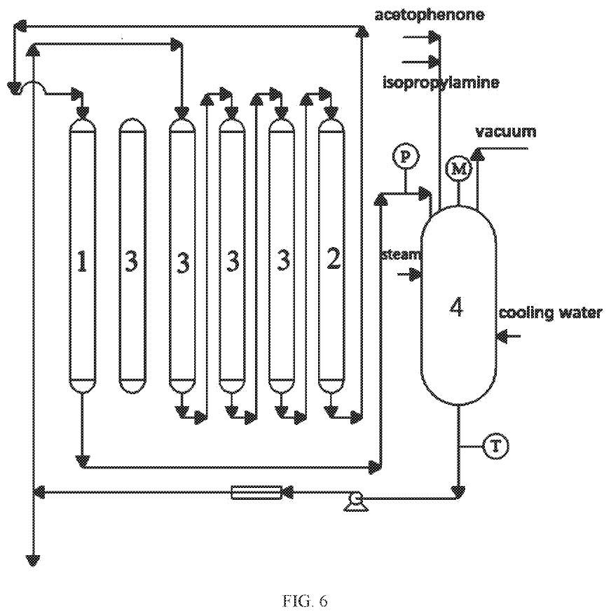

As shown in FIG. 6, when the first reaction device 1, the second reaction device 2 and several third reaction devices 3 and the circulation device 4 in the circulation loop are partially in a normal state, that is, partially in operating state, the first one of a third reaction device 3 is in an overhaul state, and the corresponding valve is closed so that the material does not flow through the first one of a third reaction device 3. At this time, the circulation loop is: the circulation device 4→the second one of a third reaction device 3→the last one of a third reaction device 3→the second reaction device 2→the first reaction device 1→circulation device 4.

It can be seen from FIG. 5 and FIG. 6 that when the same one of a third reaction device 3 is missing, the circulation direction of the circulation loop can be adjusted by opening and closing the valves, to accommodate to different production environments and production needs.

### EXAMPLE 3

This embodiment is a specific embodiment of the present invention. Using the production equipment of phenethylamine provided in the present invention, at the scale of 1000 L volume in the production system, 75 Kg of the transaminase-expressing wet cells is loaded in the middle chambers of the first reaction device 1, the second reaction device 2 and the third reaction device 3. Then, the well-stirred mixture of 800 L acetophenone, 120 Kg isopropylamine, and 100 g pyridoxal phosphate are loaded to the circulation device 4 to start the circulation reaction. The temperature range is 35-45° C., the circulation flow rate is 500-700 kg/h, and the pressure range is 0.15-0.2 Mpa. The reaction time is 20-26 h, and the product can be accumulated to 170 Kg/L in the reaction. Downstream processing can be carried out using the reaction device and method in Example 1, and unreacted acetophenone and isopropylamine can be separated and recovered. The recovered acetophenone and isopropylamine can also be loaded back into production process. The transaminase-expressing wet cells in the middle. chamber can be reused, and can still maintain more than 70% of original activity after 8 days of continuous use.

### EXAMPLE 4

This embodiment is a specific embodiment about downstream processing in the present invention, which corresponds to step S3-step S7 in embodiment 1.

1000 mL, solution of the enzymatic reaction is mixed with 180 mL of concentrated hydrochloric acid to adjust the pH to 1. The reaction mixture is distilled under reduced pressure of −0.095 Mpa to recover acetone. After the acetone is evaporated, the reaction mixture is allowed to stand for phase separation. The upper layer is the acidic aqueous phase and the lower layer is the acetophenone organic phase. After the two phases are separated in this step, the aqueous phase is about 750 mL and the organic phase is about 500 mL. The acetophenone organic phase is mixed with the alkaline wastewater to adjust pH to 7-8, then phase separation happens, and the acetophenone is recovered. The acidic aqueous phase is subject to extraction by repetitively adding 200 mL of dichloromethane each time, the dichloromethane phases are combined and concentrated below 50° C. to recover the dichloromethane. Aqueous solution of sodium hydroxide is added to the remaining organic to adjust the pH to 7-8, and the acetophenone is recovered after phase separation.

81 g of solid sodium hydroxide is added to the acidic aqueous phase after extraction to adjust the pH to 10-11. Then, this mixture is directly heated to 50° C., and isopropylamine is recovered by distillation under reduced pressure. After there is no obvious liquid flowing out, the remaining mixture is allowed to stand still for phase separation, the upper layer is crude phenethylamine, and the lower layer is alkaline wastewater. The crude phenethylamine is distilled in a distillation tower to obtain R-phenethylamine product.

The recovered acetophenone was light yellow in color, the recovery yield was 90%, and the yield of R-phenethylamine was 75%. This downstream processing achieves the stepwise recovery of acetone, acetophenone, isopropylamine, and R-phenethylamine, and recovers the solvent dichloromethane, which reduces waste generation, is conducive to resource saving and environmentally friendly.

### COMPARATIVE EXAMPLE 1

A traditional chemical method for preparing R-(+)-1-phenethylamine, referring to Chinese Patent CN103641724A, comprises the following steps: phenylacetamide and toluene were added to a tetrahydrofuran solution with zinc borohydride, and it was slowly heated to make the internal temperature reach 93° C. which was then kept for 3.5-4.5 hours with stirring; after the reaction solution was naturally cooled to room temperature, it was added to 10% hydrochloric acid, followed by filtration; the filtrate is extracted with chloroform, and the pH was adjusted to 11-12 with 20% sodium hydroxide, and it was extracted with chloroform again; the extracts were combined and dried with anhydrous MgSO4, the chloroform was recovered, and phenethylamine was obtained by distillation under reduced pressure.

### COMPARATIVE EXAMPLE 2

To a 150 mL three-necked reaction flask using mechanical stirring, 3.0 g of fresh wet cells, 2 mL of 20 mM pyridoxal phosphate aqueous solution, 7.5 mL isopropylumine, and 80 mL acetophenone were added, and the reaction was stirred at 35° C. for 24 h. The reaction was sampled for analysis, and the conversion reached only 12%. After the reaction solution was filtered, the filter cake was actually the wet cells loaded into the reaction. The above reaction setup was repeated by replacing the fresh wet cells with the filter cake. After 24 hours, the conversion reached only 3% as analyzed by HPLC, and the wet cells could not be reused normally under mechanical stirring.

### COMPARATIVE EXAMPLE 3

To a three-necked reaction flask using mechanical stirring, 3.0 g of fresh wet cells, 2 mL of 20 mM pyridoxal phosphate aqueous solution. 7.5 mL isopropylamine, 40 mL acetophenone and 40 mL water were added, and the reaction was stirred at 35° C. for 24 h. The reaction was sampled and the conversion was only 5% as analyzed by HPLC. In the downstream process of the reaction solution, the filtration rate is slow and it is difficult to realize mass production.

Compared with the traditional chemical method, the phenethylamine prepared by the present invention does not use strongly toxic organics, high temperature and pressure. It is reduced from a three-step reaction to a one-step reaction, the conversion is greatly improved, the ee value is greater than 99.5%, and the purity of the product is high. In addition, the present invention adopts a cyclic flow reaction. Compared with a mechanical stirring reaction, the conversion is high. The transaminase can be reused. After 8 days of continuous use, the transaminase retains 70% of its original activity, which greatly reduces the production cost and does not suffer the problem of slow filtration or the difficulty to scale up production.

The above are only preferred embodiments of the present invention, and do not therefore limit the implementation and protection scope of the present invention. For those skilled in the art, they should be aware that any technical solutions obtained by equivalent substitutions and obvious changes made by using the contents of the description and the illustrations of the present invention shall all be included in the protection scope of the present invention.

What is claimed is:

1. A production equipment of phenethylamine which comprising:
a first reaction device set with a transaminase;
a second reaction device, wherein the second reaction device is connected to the first reaction device, and the second reaction device is set with the transaminase;
a circulation device, wherein the circulation device is respectively connected with the first reaction device and the second reaction device:
an acetone storage device connected to the circulation device;
a centrifugal extraction device connected to the circulation device;
a phenethylamine processing module connected to the centrifugal extraction device;
phenethylamine storage device connected to the phenethylamine processing module;
wherein the first reaction device and the second reaction device have a same structure.

2. The production equipment of phenethylamine according to claim I, wherein the phenethylamine processing module comprises:
a phenethylamine inorganic acid salt separation device connected to he centrifugal extraction device:
a neutralization device connected to the phenethylamine inorganic acid salt separation device:
a phenethylamine distillation device respectively connected with tine neutralization device and the phenethylamine storage device.

3. The production equipment of phenethylamine according to claim 2, further comprising:
an acetophenone separation device connected to the centrifugal extraction device;
an acetophenone recovery device connected to the acetophenone separation device.

4. The production equipment of phenethylamine according to claim 3, further comprising:
a dichloromethane separation device respectively connected to the phenethylamine inorganic acid salt separation device and the acetophenone recovery device;
a dichloromethane recovery device connected to the dichloromethane separation device.

5. The production equipment of phenethylamine according to claim 1, further comprising:
at least one third reaction device, wherein the at least one third reaction device is respectively connected with the first reaction device and the second reaction device, and the at least one third reaction device is set with the transaminase.

6. A production method of phenethylamine using the production equipment of phenethylamine according to claim 1 comprising the following steps:
step S1: supplying acetophenone, isoproylamine, and pyridoxal phosphate to the circulation device;
step S2: circulating the acetophenone, the isopropylamine, and the pyridoxal phosphate several times in a circulation loop formed by the circulation device, the first reaction device, and the second reaction device, afterwards, obtaining acetone and crude phenethylamine;
step S3: transporting, by the circulation device., the acetone to the acetone storage device for storage, and transporting, by the circulation device, the crude phenethylamine to the centrifugal extraction device;
step S4: with an action of an inorganic acid, processing the crude phenethylamine in the centrifugal extraction device to obtain first recovered acetophenone and a phenethylamine inorganic acid salt;
step S5: transporting, by the centrifugal extraction device, the first recovered acetophenone to an acetophenone recovery device for storage, and transporting, by the centrifugal extraction device, the phenethylamine inorganic acid salt to the phenethylamine processing module;
step S6: with an action of dichloromethane and alkali sequentially, processing the crude phenethylamine in the phenethylamine processing module to obtain second recovered acetophenone and the phenethylamine, respectively;
step S7: transporting, by the phenethylamine processing module, the phenethylamine to the phenethylamine storage device for storage;
wherein the transaminase is stored in the first reaction device and the second reaction device.

7. The production method of phenethylamine according to claim 6, wherein an amino acid sequence of the transaminase shown as in SEQ ID NO: 2.

8. The production method of phenethylamine according to claim 6, wherein the step S5 further comprises:
step S51A: transporting, by the centrifugal extraction device, the first recovered acetophenone to an acetophenone separation device;
step S51B: with an action of an alkali, processing the first recovered acetophenone in the acetophenone separation device to obtain an inorganic sodium salt wastewater and acetophenone, respectively;
step S51C: transporting, by the acetophenone separation device, the acetophenone to the acetophenone recovery device for storage.

9. The production method of phenethylamine according to claim 6, wherein the step S6 further comprises:
step S61: with an action of dichloromethane, processing the crude phenethylamine in a phenethylamine inorganic acid salt separation device to obtain an aqueous solution of the phenethylamine inorganic acid salt and a mixture of dichloromethane and acetophenone, respectively;
step S62: transporting, by the phenethylamine inorganic acid salt separation device, the aqueous solution of the phenethylamine inorganic acid salt to a neutralization device, and transporting, by the phenethylamine inorganic acid salt separation device, the mixture of dichloromethane and acetophenone to a dichloromethane separation device;
step S63: with an action of an alkali, processing the aqueous solution of the phenethylamine inorganic acid salt in the neutralization device to obtain the crude phenethylamine and an inorganic sodium salt wastewater, respectively;
step S64: transporting, by the neutralization device, the crude phenethylamine to a phenethylamine distillation device;
step S65: processing the crude phenethylamine in the phenethylamine distillation device to obtain a finished product of the phenethylamine;
step S66: transporting, by the phenethylamine distillation device, the finished product of the phenethylamine to the phenethylamine storage device for storage.

10. The production method of phenethylamine according to claim 9, wherein the step S62 further comprises:
Step S621A: processing the mixture of dichloromethane and acetophenone in the dichloromethane separation device to obtain dichloromethane and acetophenone, respectively;
Step S621B: transporting, by the dichloromethane separation device, the dichloromethane to a dichloromethane recovery device for storage, and transportiming, by the dichloromethane separation device, the acetophenone to the acetophenone recovery device for storage.

11. The production method of phenethylamine according to claim 6, wherein in the step S3, the circulation device transports the crude phenethylamine and the acetone to a buffer device, the inorganic acid is transported to the buffer device; after reacting under certain conditions, the acetone and an aqueous phase are obtained and separated, the buffer device transports the acetone to the acetone storage device for storage, and the buffer device transports the aqueous phase to the centrifugal extraction device;
step S4, the aqueous phase is processed in the centrifugal extraction device to obtain the first recovered acetophenone and the phenethylamine inorganic acid salt, respectively.

12. The production method of phenethylamine according to claim 6, wherein a loading of the acetophenone, the isopropylamine, the pyridoxal phosphate, and the transaminase is as follows: the acetophenone 300-1200 L, the isopropylamine 50-240 Kg, the pyridoxal phosphate 75-220 g, and the transaminase 30-120 Kg, wherein the transaminase is transaminase-expressing wet cells, and an effective content of the transaminase in the transaminase-expressing wet cells is 1-20%
in the step S1, the mixture of the acetophenone, the isopropylamine, and the pyridoxal phosphate is preheated to 35-45° C. in the circulation device;
in the step S2, a circulation flow rate is 100-700 kg/h, the circulation flow rate refers to a flow rate in the first reaction device, a circulation reaction time is 15-26 h, and a system pressure is 0.15-0.2 Mpa;
in the step S4, a reaction temperature of the crude phenethylamine and the inorganic acid is lower than 70° C.;
in the step S4, a processing temperature of the centrifugal extraction device is 40-60° C.


SEQUENCE LISTING (2 sequences)

SEQ ID NO: 1 - DNA, 972 bp, Artificial Sequence
  The sequence is synthetized.
atggcaagcatggataaagtcttcagcggctattacgcgcgtcaaaaactgctggaacgctctgataacccgtttagcaaaggcatcgcgtacgttgaaggtaaactggttctgccgagcgacgcacgtattccgctgctggacgaaggttttatgcacagcgacgcgacctacactaccattagcgtttgggacggccgctttttccgtctggacgatcatctgcagcgtattctggaatcctgcgacaaaatgcgcctgaaatttccgctggcactgagcagcgttaaaaaaatcctggcggaaatggttgcgaaatctggtattcgcgatgcgatggtcgaagttattgttacccgcggtctgaccggcgttcgcggtcgccaaccggaagacctgtacaacaacaacatctacctgctggttctgccgtatatctggctgatggcaccggaagatcaacgtcacggcggcgaagcaattattacccgtaccgttcgtcgtaccccgccgggcgcattcgatccgaccattaaaaactttcagtggggcgatctgatcaaaggcatgttcgaagcagaagatcgcggcgcaacctatccgtttctgaccgacggcgatacccacctgaccgaaggtagcggttttaacatcgtcctggtcaaaaacggcatcatctacaccccggatcgcggcgttctggaaggtattacccgtaaaagcgttattgacgttgcacgcgcaaacagtattgatattcgcctggaagtcgtcccggttgaacaggcgtatcacagcgacgaaatcttcatgaccagcaccgctggcggtattatgccgattaccctgctggacggtcaaccggttaacggcggtcaggtaggcccgattaccaaaaagatttgggacggctactgggagatgcattacaataacgcgtatagctttccggtcgattacggtagcggttaa

SEQ ID NO: 2 - protein, 323 aa, Artificial Sequence
  The sequence is synthetized.
MASMDKVFSGYYARQKLLERSDNPFSKGIAYVEGKLVLPSDARIPLLDEGFMHSDATYTTISVWDGRFFRLDDHLQRILESCDKMRLKFPLALSSVKKILAEMVAKSGIRDAMVEVIVTRGLTGVRGRQPEDLYNNNIYLLVLPYIWLMAPEDQRHGGEAIITRTVRRTPPGAFDPTIKNFQWGDLIKGMFEAEDRGATYPFLTDGDTHLTEGSGFNIVLVKNGIIYTPDRGVLEGITRKSVIDVARANSIDIRLEVVPVEQAYHSDEIFMTSTAGGIMPITLLDGQPVNGGQVGPITKKIWDGYWEMHYNNAYSFPVDYGSG